(19) United States
(12) Patent Application Publication
(10) Pub. No.: US 2003/0014774 A1
(43) Pub. Date: Jan. 16, 2003

(54) Transgenic rice plant and its family with environmental stress resistant by proline accumulation of high level and its production

(76) Inventors: Yoshu Yoshiba, Ageo (JP); Kazuko Shinozaki, Tsukuba (JP); Kazuo Shinozaki, Kukizakimachi (JP)

Correspondence Address: Stanley P. Fisher, Reed Smith Hazel & Thomas LLP, Suite 1400, 3110 Fairview Park Drive, Falls Church, VA, 22042-4503, US

(21) Appl. No.: 10/026,767

(22) Filed: Dec. 27, 2001

(30) Foreign Application Priority Data
Jun. 8, 2001 (JP) 2001-174553

(51) Int. Cl. A01H 5/00

(52) U.S. Cl. 800/278; 800/320.2

(57) ABSTRACT

In order to obtain a transformed rice plant having an improved salinity tolerance level because of its enhanced proline accumulating ability, a P5CS (Δ¹-pyrroline-5-carboxylate (P5C) synthetase) gene of rice or a P5CS gene of Arabidopsis thanliana and the antisense (reverse DNA sequence-containing) gene of a ProDH (Proline dehydrogenase) are introduced into a rice plant by using a genetic engineering technology.

### BACKGROUND OF THE INVENTION

The present invention relates to a rice plant having a high level of proline accumulating ability, and improved salinity-tolerance, drought-tolerance, and low temperature-tolerance, and its production method.

It is known that, for several plants including halophytes, when the plants are subjected to a high salinity stress or a drought stress, they accumulate proline, which is one of amino acids, in their cytoplasms. This is considered useful for regulating the osmotic pressure in the plant cytoplasm, or inhibiting the degradation of a functional protein due to the stress. The proline in a plant is synthesized from a glutamic acid by two enzymes of a Δ¹-pyrroline-5-carboxylate (P5C) synthetase (P5CS) and a P5C reductase. On the other hand, proline is degraded into a glutamic acid by the two enzymes of a proline dehydrogenase (ProDH) and a P5C dehydrogenase.

When each of the aforesaid plants is subjected to a water stress (the state in which water is difficult to absorb) such as a high salinity stress or a drought stress, the expression level of the P5CS gene is increased to activate the P5CS. However, the P5CR activity and the gene expression are constant at a low level. Further, the gene expression and the enzyme activity related to metabolism are also in the inhibited states. However, once the water stress has been removed, conversely, this time, the gene expression and enzyme activity related to biosynthesis are inhibited, so that the expression of the ProDH gene is rapidly induced, and the enzyme activity is also enhanced. As a result, the proline accumulated in the cytoplasm is rapidly metabolized to a glutamic acid.

From the foregoing description, it is considered that the P5CS becomes rate-limiting for proline synthesis under a water stress. Whereas, the ProDH becomes rate-limiting for proline metabolism after releasing the water stress (Yoshida et al., Plant Cell Physiol, 38: 1095-1102 (1997)).

### SUMMARY OF THE INVENTION

It is predicted that food shortage due to an expansion of the saline soil area caused by drought and semi-drought with the deterioration of global environment, and population growth will become increasingly more serious in the future. Researches have been pursued in diversified fields respectively on the breeding of crop plants resistant to a high salinity stress, a drought stress, and a low temperature stress (the state in which water is difficult to absorb) as those playing an important role in solving the world food problem, and the results are expected to be promising.

It is an object of the present invention to provide a rice plant which has a high proline accumulating ability, and accordingly has improved salinity-tolerance, drought-tolerance, and low temperature-tolerance by focusing attention on the importances of a Δ¹-pyrroline-5-carboxylate (P5C) synthetase (P5CS) and a proline dehydrogenase (ProDH) which are the rate-limiting enzymes related to synthesis and metabolism of proline in plants, and regulating the expression of genes for the enzymes with a gene recombination technology, and its production method.

The P5CS gene related to proline synthesis is introduced to be overexpressed; the antisense (reverse DNA sequence-containing) gene of the ProDH gene related to the metabolism is introduced to inhibit the degradation of proline; or both the P5CS gene and the antisense gene of the ProDH gene are introduced to promote the proline synthesis while inhibiting the degradation of proline. As a result, proline is accumulated with a high concentration in the cells of rice and a rice plant.

In the present invention, by accumulation of proline at a high concentration, it becomes possible to perform molecular breeding of rice and a rice plant having salinity-tolerance, drought-tolerance, or low temperature-tolerance.

Heretofore, there is known no report that an increase in concentration of proline as an osmoprotectant is allowed by synthesis promotion and degradation inhibition in rice and a rice plant. The inventors of the present invention have focused attention on the importances of the P5CS gene and the ProDH gene. Then, in order to solve novel technical problems which have not been known in the prior art, they have conducted studies from various fields including the study on the selection of the rice variety into which the gene is easily introduced, the study for improving the callus formation rate, the study on the construction of a vector for introducing the gene for rice, and the like. In consequence, they have provided novel technical elucidation, resulting in the completion of the present invention.

In the present invention, there are provided a rice plant transformed by introducing therein the proline synthesis gene and the antisense gene of the proline metabolism gene derived from rice or Arabidopsis thaliana individually or in combination, and its production method.

In the rice plant of the present invention, either or both of the gene encoding the synthetase protein of proline which is one of amino acids and the antisense gene of the proline dehydrogenage have been introduced. With this construction, it is possible to implement a rice plant having improved salinity-tolerance, drought-tolerance, and low temperature-tolerance. Further, the mature rice seeds gathered from the rice plant of the present invention, particularly the rice seeds are characterized by keeping a high proline accumulating ability over a plurality of generations.

Further, the present invention is targeted for rice and rice plants. The targets have no particular restriction as long as they are the plants belonging to the rice plants. Examples of the plants belonging to the rice plants include rice, corn, wheat, barley, rye, turf, millet, and barn grass. In particular, the present invention can be more preferably applied to rice.

### BRIEF DESCRIPTION OF THE DRAWINGS

FIGS. 1A to 1D are diagrams respectively showing the vectors for rice in which proline synthesis-related enzyme P5CS genes and proline metabolism-related enzyme ProDH genes, and antisense genes thereof have been respectively incorporated;

FIG. 2 is a graph showing the amount of proline accumulated in rice lines under no stress in which the vectors shown in FIGS. 1A to 1D have been respectively introduced by genetic engineering; and

FIG. 3 is a graph showing the salinity-tolerance of each of the transgenic rice lines in which the proline-related genes have been respectively incorporated shown in FIG. 2.

### DETAILED DESCRIPTION OF THE PREFERRED EMBODIMENTS

In rice plants of examples of the present invention, either or both of the proline (osmoprotectant) synthesis gene and the antisense gene of the proline motabolism derived from rice or Arabidopsis thaliana gene have been introduced for transformation.

Examples of one type of gene to be introduced to the rice plants of the examples of the present invention include: (1) a P5CS (Δ¹-pyrroline-5-carboxylate (P5C) synthetase) gene of rice containing the sequence (DNA sequence and amino acid sequence) according to SEQ ID No. 1; (2) a P5CS (Δ¹-pyrroline-5-carboxylate (P5C) synthetase) gene of Arabidopsis thaliana containing the sequence (DNA sequence and amino acid sequence) according to SEQ ID N2; and (3) the antisense (reverse DNA sequence-containing) gene of the ProDH (proline dehydrogenase) gene of Arabidopsis thaliana containing the sequence (DNA sequence and amino acid sequence) according to Seq ID NO. 3.

Examples of the two types of genes to be introduced into the rice plants of the examples of the present invention include:

(1) Two genes of the P5CS (Δ¹-pyrroline-5-carboxylate (P5C) synthetase) of rice containing the sequence according to SEQ ID NO. 1 or the P5CS gene of Arabidopsis thaliana containing the sequence according to SEQ ID NO. 2, and the antisense (reverse DNA sequence-containing) gene of the ProDH (proline dehydrogenase) gene of Arabidopsis thaliana containing the sequence according to SEQ ID NO. 3; and

(2) Tandemly connected two genes of the P5CS (Δ¹-pyrroline-5-carboxylate (P5C) synthetase) gene of rice containing the sequence according to SEQ ID NO. 1 or the P5CS gene of Arabidopsis thaliana containing the sequence according to SEQ ID NO. 2, and the antisense (reverse DNA sequence-containing) gene of the ProDH (proline dehydrogenase) gene of Arabidopsis thaliana containing the sequence according to SEQ ID NO. 3.

In each of the vectors to be used in the examples of the present invention, there is incorporated any one gene of the P5CS (Δ¹-pyrroline-5-carboxylate (P5C) synthetase) gene of rice containing the sequence according to SEQ ID NO. 1, the P5CS gene of Arabidopsis thaliana containing the sequence according to SEQ ID NO. 2, and the antisense (reverse DNA sequence-containing) gene of the ProDH (proline dehydrogenase) gene of Arabidopsis thaliana containing the sequence according to SEQ ID NO. 3. Alternatively, there are incorporated two genes of the P5CS gene of rice or Arabidopsis thaliana, and the aforesaid antisense gene in tandemly connected relation to each other.

The rice plants of the examples of the present invention can be obtained by, for example, any of the following methods.

(1) The aforesaid vector is introduced into the calli derived from a rice plant, and the calli are grown. Then, a plant body is regenerated from the calli;

(2) The aforesaid vector is introduced into the protoplast derived from a rice plant, and a plant body is regenerated from the colony obtained by growing the protoplast; and

(3) Crossing with the rice plants obtained by introducing the vector therein by genetic engineering is carried out.

Examples of the production method of the rice plants of the examples of the present invention include the following methods:

(1) The aforesaid vector is introduced into the calli derived from a rice plant by using Agrobacterium tumefaciens, and the calli are grown. Then, a plant body is regenerated from the calli;

(2) The aforesaid vector is introduced into the protoplast derived from a rice plant by electroporation, and a plant body is regenerated from the colony obtained by growing the protoplast; and

(3) Crossing with the rice plants obtained by introducing the vector therein by genetic engineering is carried out.

These production methods provide a rice plant having a high proline accumulating ability, and having improved salinity-tolerance, drought-tolerance, and low temperature-tolerance levels.

Further, mature seeds gathered from the rice plants of the examples of the present invention, particularly the rice seeds will maintain their high proline accumulating abilities over a plurality of generations.

The rice plants of the examples of the present invention and its production method will be described in details by way of embodiments thereof by using rice as a typical example step by step below. It is needless to say that the steps described below are applicable to other rice plants than rice with or without changing the various conditions.

(Gene Cloning)

First, a mRNA is extracted from a rice seedling. A cDNA is synthesized by using the mRNA. The cDNA is combined with a vector made of a plasmid or a phage, and introduced into E. coli to prepare a recombinant DNA. The resulting transformant in which the recombinant DNA has been introduced is subjected to screening by plaque hybridization using the P5CS gene from Arabidopsis thaliana as a probe. The sequences of the P5CS genes from rice and Arabidopsis thaliana have been already reported (Yoshiba et al., Plant J. (1995) 7:751-760, and Igarashi et al., Plant Mol. Biol. (1997) 33:857-865). Based on these reports, appropriate primers are designed, and subjected to screening by PCR to select a target transformant. A target plasmid is isolated from the transformant obtained. If required, it is cut with an appropriate restriction enzyme, and subjected to subcloning in a plasmid vector for cloning. It is also possible to subject the P5CS gene of Arabidopsis thaliana to cloning in the same manner as with rice. However, as a sample from which a mRNA is to be extracted, the one subjected to a high salinity stress (immersed in a 250 mM NaCl solution or the like) or the one subjected to a drought stress treatment is more preferable than the one bred under a normal environment. This is because the P5CS gene is induced in response to a water stress such as a high salinity stress or a drought stress (Yoshiba et al., Plant J. (1995) 7: 751-760, Igarashi et al., Plant Mol. Biol. (1997) 33: 857-865, and Yoshiba et al., Plant Cell Physiol. (1997) 38: 1095-1102).

On the other hand, it is also possible to subject the ProDH gene of Arabidopsis thaliana (its sequence has already been reported in Kiyosue et al., Plant Cell (1996) 8:1323-1335) to cloning in the foregoing manner. However, as the sample from which a mRNA is to be extracted, there may be used the one which has been subjected to a drought stress (about 10-hour treatment), then immersed in water again, and allowed to absorb water, the one which has been immersed in a proline solution, and allowed to absorb proline, or the like. This is due to the following fact. Namely, the ProDH gene is inhibited from its expression under a water stress, and the gene expression is induced by a high concentration of proline (Kiyosue et al., Plant Cell (1996) 8: 1323-1335, and Yoshiba et al., Plant Cell Physiol. (1997) 38: 1095-1102).

If the samples as described above are used, it is possible to isolate the P5CS gene and the ProDH gene not only from rice or Arabidopsis thaliana but also from other rice plants.

(Construction of Gene Introduction Vector)

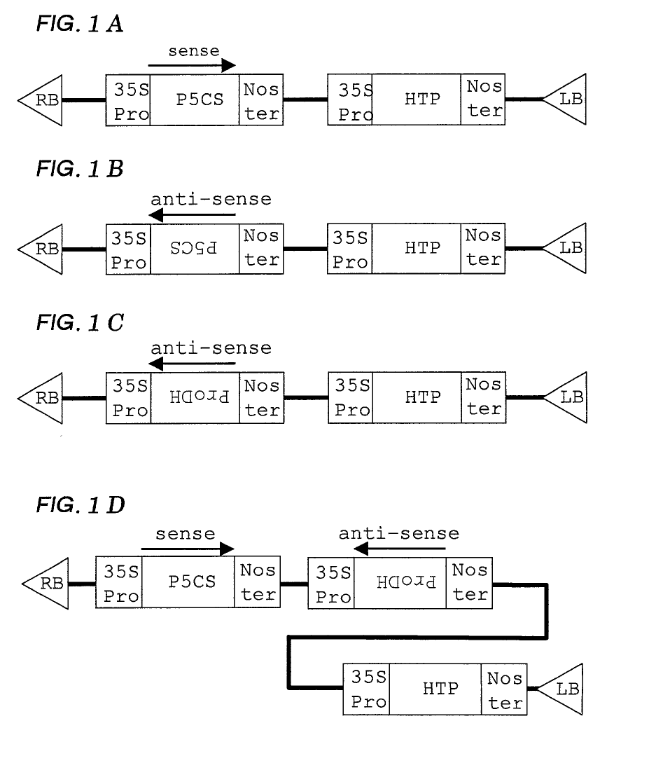

Respective P5CS genes and ProDH genes subjected to cloning are cut from plasmids with appropriate restriction enzymes, and, as shown in FIGS. 1A to 1D, each is combined behind the 35S promoter of a cauliflower mosaic virus of a vector for rice obtained by modifying a pBI vector. In FIGS. 1A to 1D, RB denotes the right border, 35SPro denotes the promoter of a cauliflower mosaic virus, P5CS denotes the proline synthesis-related enzyme gene of rice or Arabidopsis thaliana, ProDH denotes proline metabolism-related enzyme gene of Arabidopsis thaliana, Noster denotes the terminator of a nopaline synthetase gene, HTP denotes a hygromycine resistant gene, and LB denotes the left border. Whereas, each of the arrows indicates the orientation of the sense of each gene.

In FIGS. 1A to 1D, FIG. 1A is a diagram showing an example of the vector (construct) so constructed that the sequence in the order of RB-35SPro-P5CS-Noster-35SPro-HTP-Noster-LB has been achieved. FIG. 1B is a diagram showing an example in which, with respect to FIG. 1A, the same sequence in the order of RB-35SPro-P5CS-Noster-35SPro-HTP-Noster-LB as in the construct of FIG. 1A has been achieved, but the gene P5CS has been sequenced in antisense orientation. FIG. 1C is a diagram showing an example in which the gene ProDH has been sequenced in antisense orientation, and substituted for the gene P5CS of the construct of FIG. 1A, to construct a vector with a sequence in the order of RB-35SPro-ProDH (antisense)-Noster-35SPro-HTP-Noster-LB. FIG. 1D is a diagram showing an example in which, to the construct of FIG. 1A, the gene ProDH has been further sequenced in antisense orientation, and the construct shown in FIG. 1C has been further connected thereto in tandem, to construct a vector with a sequence in the order of RB-35SPro-P5CS-Noster-35SPro-ProDH (antisense)-Noster-35SPro-HTP-Noster-LB.

The 35S promoter is well known as a promoter which is strong and invariably induces the gene expression in any tissue. As for the orientation in which the gene is incorporated, the P5CS gene is connected in the sense orientation, and the ProDH gene in the antisense orientation.

Then, each vector to which each of the genes has been connected is introduced into Agrobacterium tumefaciens EHA 101 by electroporation. The Agrobacterium tumefaciens in which each construct (FIGS. 1A to 1D) has been introduced is cultured and grown in a YEP medium containing Bacto Pepton (10 g/l), Bacto Yeast Extract (10 g/l), sodium chloride (5 g/l), 1M magnesium chloride (2 ml/l), and hygromycine B (50 mg/l) at 28° C. Gene introduction is carried out by infecting the callus cell of rice with the Agrobacterium tumefaciens into which each construct (FIGS. 1A-1D) has been introduced. The construct D is so designed that the two genes (the P5CS gene and the ProDH gene) are connected to each other in tandem to be simultaneously introduced. However, even if the constructs A and C are mixed for coinfection, it is also possible obtain the same effects as with the construct D.

Incidentally, a HPT (hygromycine resistant) gene is connected to each construct. This is for efficiently selecting the cell and plant body transformed for the basic research on analysis of the effects of the introduced genes. Therefore, the HPT gene is not required to be incorporated therein for actual cultivation on the salt damaged land or the dry land.

(Induction of Rice Calli for Gene Introduction)

Mature rice seeds are sterilized with 70% ethyl alcohol for 10 minutes, and with 3% sodium hypochlorite for 1 hour after stripping the hulls therefrom. After sterilization, the seeds are washed with sterilized water 3 times, and bedded on a pH 5.8 N6 medium (2N6 medium) containing 1 g/l casamino acid, 30 g/l sucrose, 2 mg/l 2,4-dichlorophenoxyacetic acid, and 2 g/l Gelrite, and cultured at 28° C. in the dark for 3 to 5 weeks.

(Gene Introduction into Rice Calli)

Out of the rice calli induced in the foregoing manner, the ones with a size of 1 to 3 mm are bedded on the 2N6 medium again, and cultured at 28° C. in the dark for 3 to 4 days. As a result, it is possible to enhance the division activity of the callus cell. The gene introduction is carried out by mixing the cultured calli and a solution of each construct-introduced Agrobacterium tumefaciens grown in the YEP medium (the solution diluted so that the concentration of the bacteria is 0.1 as determined at OD 660 nm) for infection. Thereafter, the calli are cultured at 25° C. in the dark for 3 days. After cultivation, the calli are washed and sterilized several times by a cefotaxime aqueous solution with a concentration of 1 mg/4 ml to remove extra bacteria attached to the surfaces of the calli, and cleaned with a sterilized kim towel or the like. Subsequently, it is bedded on a 2N6 medium (secondary selection medium) containing 250 mg/l cefotaxime and 10 mg/l hygromycine B, and cultured at 28° C. in the dark for 1 week.

(Selection of Transformed Calli and Regeneration of Plant Body)

The calli cultured in the medium containing cefotaxime is bedded on a medium (secondary selection medium) in which the content of hygromycine B has been increased to 30 mg/l, and cultured at 28° C. in the dark for 3 weeks. Thereafter, the calli are transferred to a pH 5.8 MS medium (regeneration induction medium) containing 30 g/l sucrose, 30 g/l sorbitol, 2 g/l casamino acid, 11 g/l MES buffer, 2 mg/l NAA, 1 mg/l kinetin, 250 mg/l cefotaxime, 30 mg/l hygromycine B, and 4 g/l Gelrite, and cultured in the bright place at 28° C. for 3 week. The gene-introduced calli form a green spot, from which shoots and roots are regenerated. The regenerated calli are further transferred to a pH 5.8 MS medium (plant body formation medium) containing 30 g/l sucrose, 250 mg/l cefotaxime, 30 mg/l hygromycine B, and 8 g/l agar, from which plant hormones have been removed, and cultured in the bright place at 28° C. for several weeks. In consequence, the plant body is bred more largely.

(Breeding of Transformed Rice Plant Body and Seed Formation)

Upon having grown to a seedling height of about 4 to 5 cm in a petri dish, the regenerated rice is transferred to a planter in which the soil for raising-seedling is placed. Then, it is bred in an artificial climate system with an illuminance of about 20,000 lx under a temperature condition of 28° C. until the fourth leaf to the fifth leaf develop. Subsequently, the seedling is further transferred into a pot containing the soil into which a fertilizer has been appropriately added, and bred in a greenhouse until the seeds ripen. Assuming that the present generation of the plant body regenerated is of the T0 generation, and that the seeds obtainable from this plant body is of the T1 generation, the ones of the T2 to T3 generations are bred. When they are cultivated in an actual farm land, they are required to be commercialized after carrying out the various safety evaluation tests over further generations, and confirming the safety.

(Extraction of Proline from Transformed Rice and Concentration Measurement Thereof)

Proline is extracted from the leaves of the seedling (whose forth leaf has developed) of the transformed rice of the T2 generation or the T3 generation. The leaves of the rice seedling bred in the artificial climate system are cut off in an amount of about 200 mg by scissors or the like. Then, in a mortar, liquid nitrogen is added thereto, and the leaves are ground into powder. The resulting sample in powder form is mixed with pure water, and further milled by means of a homogenizer or the like. The milled sample is heated at 97° C. for 6 minutes, and then ice cooled. The sample is then centrifuged at about 17,000×G for 10 minutes at 4° C. to separate the supernatant. To the supernatant obtained, a trichloroacetic acid is added and mixed so that the final concentration is 5%. The resulting mixture is then centrifuged at about 17,000×G for 10 minutes at 4° C. again to precipitate protein. Proline as an osmoprotectant is contained in the supernatant at this step, and the concentration thereof is determined by means of high performance liquid chromatography (HPLC). The qualitative determination of proline is carried out in the following manner. The solutions in which various amino acids have been dissolved to a given concentration are previously determined by HPLC. The amount of proline contained in the leaf of an actual transgenic rice is determined based on the retention times.

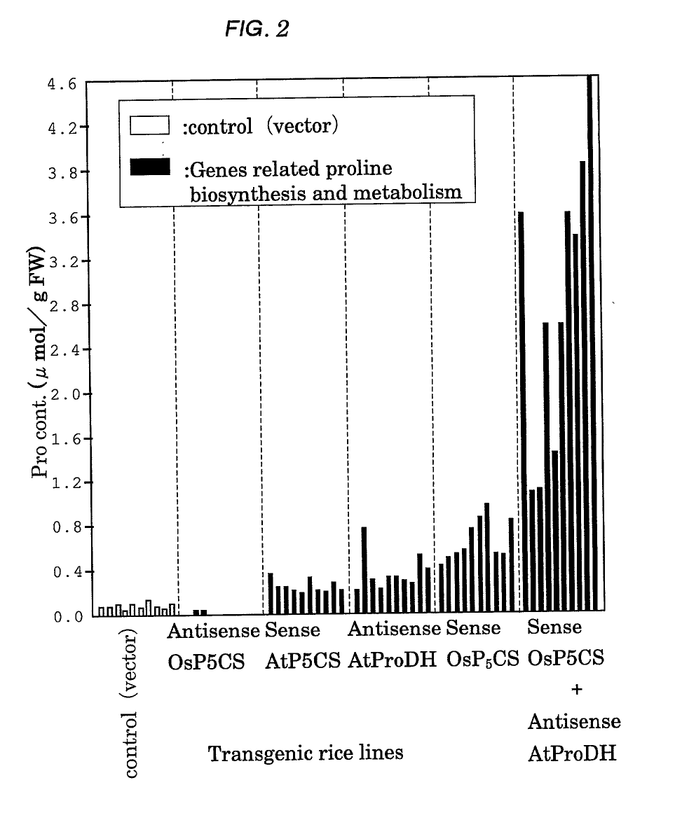

FIG. 2 shows the proline content of each of the transgenic rice lines under no stress into which various genes have been introduced. The hollow graphs in the leftmost column represent control samples into which proline-related genes have not been incorporated. Whereas, the solidly shaded graphs in the right-hand five columns denote respective transgenic rice lines into which proline-related genes have been incorporated. It is indicated that the proline content varies according to the type of the gene introduced.

There is observed almost no accumulation for each sample in which the P5CS gene (OsP5CS) of rice has been introduced in antisense orientation (FIG. 1B) in the second column from left. For each sample in which the P5CS gene (AtP5CS) of Arabidopsis thaliana has been introduced in sense orientation (FIG. 1A) in the third column from left, there is observed an increase in amount of proline accumulated over the control samples. Similarly, for each sample in which the ProDH gene (AtProDH) of Arabidopsis thaliana has been introduced in antisense orientation (FIG. 1C) and each sample in which the P5CS gene (OsP5CS) of rice has been introduced in sense orientation (FIG. 1A) in the fourth and fifth columns from left, respectively, there are observed increases in amount of proline accumulated over the control sample. In contrast to these, for each sample in which the P5CS gene (OsP5CS) of rice has been introduced in sense orientation, and the ProDH gene (AtProDH) of Arabidopsis thaliana in antisense orientation in the rightmost column, there is observed a considerably larger amount of proline accumulated (100 times or more with respect to the control sample for the case where the amount of proline accumulated is larger) as compared with each of the aforesaid samples in which one type of gene has been introduced. Then, it is indicated that each sample of OsP5CS (in the fifth column from left) is slightly more effective for proline accumulation than each sample of AtP5CS (in the third column from left) among the samples in which genes have been introduced in sense orientation.

(Salinity Tolerance Test and Improvement of Salinity Tolerance of Transgenic Rice)

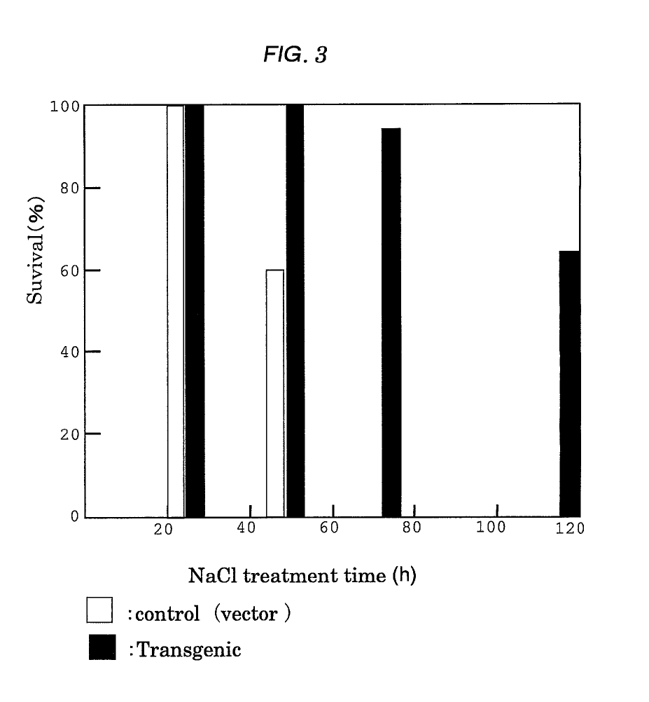

FIG. 3 shows the results of a salinity tolerance test performed at a 250 mM concentration (about half the salt concentration of sea water) by using several lines of the transgenic rice for which proline accumulation has been observed shown in the right hand four columns of FIG. 2. The hollow graphs denote the control samples in which proline related genes have not been incorporated. Whereas, the solidly shaded graphs denote the transgenic rice samples. The salinity tolerance test was carried out in accordance with the testing method using known survival rates as indexes (Japanese Published Unexamined Patent Application No. Hei 09-266726, title of the invention: evaluation of salt resistance of plant). It has been shown that the control samples in which proline-related genes have not been introduced die 5 days after a salt treatment, while the transgenic rice samples which accumulate proline show high survival rates, i.e., 95% for the third day, and 65% even after the five-day treatment. This indicates that the salinity tolerance can be improved by transforming rice, and thereby enhancing the proline accumulating ability thereof.

Therefore, if the gramineous crop produced according to the present invention is subjected to breeding by further pursuing detailed analysis such as the safety evaluation thereon, it becomes capable of being cultured in the salt accumulated soil or the desertified soil. Therefore, food productivity can be expected to be improved. Further, it can be largely expected that the crop plant is also capable of coping with the population growth in developing countries.

In accordance with the present invention, it has become possible to produce a transgenic rice plant having an enhance proline accumulating ability. Further, for the rice plant produced by the method of the present invention, the amount of proline accumulated therein has been increased, so that it has become possible to improve the salinity tolerance level thereof.

What is claimed is:

1. A rice plant in which a P5CS (Δ¹-pyrroline-5-carboxylate (P5C) synthetase) gene of rice containing the sequence according to SEQ ID NO. 1 has been introduced.

2. A rice plant in which a P5CS (Δ¹-pyrroline-5-carboxylate (P5C) synthetase) gene of Arabidopsis thanliana containing the sequence according to SEQ ID NO. 2 has been introduced.

3. A rice plant in which the antisense (reverse DNA sequence-containing) gene of a ProDH (Proline dehydrogenase) gene of Arabidopsis thanliana containing the sequence according to SEQ ID NO. 3 has been introduced.

4. A rice plant in which a P5CS gene of rice containing the sequence according to SEQ ID NO. 1, or a P5CS gene of Arabidopsis thanliana containing the sequence according to SEQ ID NO. 2, and the antisense gene of a ProDH gene of Arabidopsis thanliana containing the sequence according to SEQ ID NO. 3 have been introduced.

5. A rice plant in which a P5CS gene of rice containing the sequence according to SEQ ID NO. 1, or a P5CS gene of Arabidopsis thanliana containing the sequence according to SEQ ID NO. 2, and the antisense gene of a ProDH gene of Arabidopsis thanliana containing the sequence according to SEQ ID NO. 3 have been introduced in tandemly connected relation to each other.

6. A vector in which any of a P5CS gene of rice containing the sequence according to SEQ ID NO. 1, a P5CS gene of Arabidopsis thanliana containing the sequence according to SEQ ID NO. 2, and the antisense gene of a ProDH gene of Arabidopsis thanliana containing the sequence according to SEQ ID NO. 3 has been introduced, or said P5CS gene of rice or Arabidopsis thanliana and said antisense gene of said ProDH gene of Arabidopsis thanliana have been introduced in tandemly connected relation to each other.

7. A rice plant obtained by introducing said vector according to claim 6 into calli derived from a rice plant to grow said calli, and then regenerating a plant body from said calli.

8. A rice plant obtained by introducing said vector according to claim 6 into a protoplast derived from a rice plant, growing said protoplast to obtain a colony, and then regenerating a plant body from said colony.

9. A rice plant obtained by crossing with a rice plant obtained by introducing said vector according to claim 6 therein by genetic engineering, wherein said vector according to claim 6 has been introduced.

10. The rice plant according to any of claims 1 to 9, wherein said rice plant is rice.

11. A seed of a rice plant collected from said rice plant according to any of claims 1 to 9.

12. A seed of the rice plant according to any of claims 1 to 9, wherein said rice plant is rice, said seed having been collected from said rice.

13. A production method of a rice plant, comprising: introducing said vector according to claim 6 into calli derived from a rice plant by using Agrobacterium tumefaciens to grow said calli; and then regenerating a plant body from said calli.

14. A production method of a rice plant, comprising: introducing said vector according to claim 6 into a protoplast derived from a rice plant by electroporation, and growing said protoplast to obtain a colony, and regenerating a plant body from said colony.

15. A production method of a rice plant, comprising: crossing with a rice plant obtained by introducing said vector according to claim 6 by genetic engineering, and introducing said vector according to claim 6 therein.


SEQUENCE LISTING (3 sequences)

SEQ ID NO: 1 - DNA, 2549 bp, Oryza sativa L.
  CDS 99..2249
  ref <301>: AUTHORS: Yumiko Igarashi, Yoshu Yoshiba, Yukika Sanada, Kazuko Yamaguchi-Shinozaki, Keishiro Wada, Kazuo Shinozaki
  ref <302>: TITLE: Characterization of the gene for 1-pyrroline-5-carboxylate synthetase and correlation between the expression of the gene and salt tolerance in Oryza sativa L.
  ref <303>: JOURNAL: Plant Molecular biology
  ref <304>: VOLUME: 33
  ref <306>: PAGES: 857-865
  ref <307>: DATE: 1996-12-03
  ref <308>: DATABASE ACCESSION NUMBER: D49714
  ref <309>: DATABASE ENTRY DATE: 1995-03-16
gcggctgcggcggcaaggcggcgagacgtgggagagggatttacaggtagagggagagggtggaggaggagaggctgaggctaggaagcggtttcgccatggcgagcgtcgacccgtcccggagcttcgtgagggacgtgaagcgcgtcatcatcaaggtgggcactgcagttgtctccagacaagatggaagattggctttgggcagggttggagctctgtgcgagcaggttaaggaactgaactctttaggatacgaagtgattttggtcacctcaggtgctgttggagtggggcgacagcgacttaggtaccggaagcttgtcaatagcagctttgctgatctgcaaaagccacagatggagttagatggaaaggcttgtgccgctgttggtcagagtggactgatggctctttacgatatgttgtttaaccaactggatgtctcgtcatctcaacttcttgtcaccgacagtgattttgagaacccaaagttccgggagcaactcactgaaactgttgagtcattattagatcttaaagttataccaatatttaatgaaaatgatgccatcagcactagaaaggctccatatgaggattcatctggtatattctgggataatgacagtttagcaggactgttggcactggaactgaaagctgatctccttattctgctcagtgatgtggatgggttgtatagtggtccaccaagtgaaccatcatcaaaaatcatacacacttatattaaagaaaagcatcagcaagaaatcacttttggagacaaatctcgtgtaggtagaggaggcatgacagcaaaagtgaaggctgctgtcttggcttcaaatagcggcacacctgtggttattacaagtgggtttgaaaatcggagcattcttaaagttcttcatggggaaaaaattggtactctctttcacaagaatgcgaatttgtgggaatcatctaaggatgttagtactcgtgagatggctgttgccgcaagagattgttcaaggcatctacagaatttgtcatcagaggaacgaaaaaagatattgctagatgttgcagatgctttggaggcaaatgaggatttaataaggtctgagaatgaagctgatgtagctgcggcccaagttgctggatatgagaagcctttggttgctagattgactataaaaccaggaaagatagcaagccttgcaaaatctattcgtacccttgcaaatatggaagaccctataaaccagatacttaaaaagacagaggttgctgatgatttagttcttgagaaaacatcttgcccattaggtgttctcttaattgtttttgagtcccgacctgatgccttggttcagattgcatctttggcaattcgaagtggtaatggtcttctcctaaaaggtggaaaagaagctatcagatcaaacacgatattgcataaggttataactgatgctattcctcgtaatgttggtgaaaaacttattggccttgttacaactagagatgagatcgcagatttgctaaagcttgatgatgtcattgatcttgtcactccaagaggaagtaataagcttgtctctcaaatcaaggcgtcaactaagattcctgttcttgggcatgctgatggtatatgccacgtatatattgacaaatcagctgacatggatatggcaaaacttattgtaatggatgcaaaaactgattacccagcagcctgcaatgcaatggagaccttactagttcataaggatcttatgaagagtccaggccttgacgacatattagtagcactaaaaacagaaggagttaatatttatggtggacctattgcgcacaaagctctgggatttccaaaagctgtttcatttcatcatgagtatagttctatggcctgcactgttgagtttgttgatgatgttcaatcagcaattgaccatattcatcgttatggaagtgctcatacagattgtatcgtcactacagatgataaggtagcagagacttttctacgcagagttgatagtgctgctgtatttcataatgcaagtacgagattctctgatggggctcgttttggattgggtgctgaggttggcataagcacagggcgtatccatgcccgtggaccagtgggtgttgaaggtctcttaactacacgatggatcttgcgaggacgtgggcaagtggtgaatggtgacaaggatgtcgtgtacacccataagagtcttcctttgcaatgaggtcaaatgctccttttagcctgttcaggagtaggtgaatatccttttaagaatggattgactactttattttgtcatcttgtacaagcatcttattgcggcattccgatggattattgattttgggggttcccactttcaaatgtgacaccaaaaataaattcatcagttctgagagcaagattttggaggttcagcttctccatgtaataagtaaattcagttctgagaacttgtgtaccaacgcgctatgttgcttgtaatgagcgatactaacatctgtgattgcacatatactaa

SEQ ID NO: 2 - DNA, 2571 bp, Arabidopsis thaliana
  CDS 107...2260
  ref <301>: AUTHORS: Yoshu Yoshiba, Tomohiro Kiyasue, Takeshi Katagiri, Hiroko Ueda, Tsuyoshi Mizoguchi, Kazuko Yamaguchi-Shinozaki, Keishiro Wada, Yoshinori Harada, Kazuo Shinozaki
  ref <302>: TITLE: Correlation between the induction of a gene for 1- pyrroline-5-carboxylate synthetase and the accumulation of proline in Arabidopsis thaliana under osmotic stress.
  ref <303>: JOURNAL: The Plant Journal
  ref <304>: VOLUME: 7
  ref <305>: ISSUE: 5
  ref <306>: PAGES: 751-760
  ref <307>: DATE: 1995-01-20
  ref <308>: DATABASE ACCESSION NUMBER: D32138
  ref <309>: DATABASE ENTRY DATE: 1994-07-12
ctgatatttattttcttaccttaaatacgacggtgcttcactgagtccgactcagttaactcgttcctctctctgtgtgtggttttggtagacgacgacgacgataatggaggagctagatcgttcacgtgcttttgccagagacgtcaaacgtatcgtcgttaaggttgggacagcagttgttactggaaaaggtggaagattggctcttggtcgtttaggagcactgtgtgaacagcttgcggaattaaactcggatggatttgaggtgatattggtgtcatctggtgcggttggtcttggcaggcaaaggcttcgttatcgacaattagtcaatagcagctttgcggatcttcagaagcctcagactgaacttgatgggaaggcttgtgctggtgttggacaaagcagtcttatggcttactatgagactatgtttgaccagcttgatgtgacggcagctcaacttctggtgaatgacagtagttttagagacaaggatttcaggaagcaacttaatgaaactgtcaagtctatgcttgatttgagggttattccaattttcaatgagaatgatgctattagcacccgaagagccccatatcaggattcttctggtattttctgggataacgatagcttagctgctctactggcgttggaactgaaagctgatcttctgattcttctgagcgatgttgaaggtctttacacaggccctccaagtgatcctaactcaaagttgatccacacttttgttaaagaaaaacatcaagatgagattacattcggcgacaaatcaagattagggagagggggtatgactgcaaaagtcaaagctgcagtcaatgcagcttatgctgggattcctgtcatcataaccagtgggtattcagctgagaacatagataaagtcctcagaggactacgtgttggaaccttgtttcatcaagatgctcgtttatgggctccgatcacagattctaatgctcgtgacatggcagttgctgcgagggaaagttccagaaagcttcaggccttatcttcggaagacaggaaaaaaattctgcttgatattgccgatgcccttgaagcaaatgttactacaatcaaagctgagaatgagttagatgtagcttctgcacaagaggctgggttggaagagtcaatggtggctcgcttagttatgacacctggaaagatctcgagccttgcagcttcagttcgtaagctagctgatatggaagatccaatcggccgtgttttaaagaaaacagaggtggcagatggtcttgtcttagagaagacctcatcaccattaggcgtacttctgattgtttttgaatcccgacctgatgcacttgtacagatagcttcacttgccatccgtagtggaaatggtcttctgctgaagggtggaaaggaggcccggcgatcaaatgctatcttacacaaggtgatcactgatgcaattccagagactgttgggggtaaactcattggacttgtgacttcaagagaagagattcctgatttgcttaagcttgatgacgttatcgatcttgtgatcccaagaggaagcaacaagcttgttactcagataaaaaatactacaaaaatccctgtgctaggtcatgctgatggaatctgtcatgtatatgtcgacaaggcttgtgatacggatatggcaaagcgcatagtttctgatgcaaagttggactatccagcagcctgtaatgcgatggaaacccttcttgtgcataaggatctagagcagaatgctgtgcttaatgagcttatttttgctctgcagagcaatggagtcactttgtatggtggaccaagggcaagtaagatactgaacataccagaagcacggtcattcaaccatgagtactgtgccaaggcttgcactgttgaagttgtagaagacgtttatggtgctatagatcacattcaccgacatgggagtgcacacacagactgcattgtgacagaggatcacgaagttgcagagctattccttcgccaagtggatagcgctgctgtgttccacaacgccagcacaagattctcagatggtttccgatttggacttggtgcagaggtgggggtaagcacgggcaggatccatgctcgtggtccagtcggggtcgaaggattacttacaacgagatggataatgagaggaaaaggacaagttgtcgacggagacaatggaattgtttacacccatcaggacattcccatccaagcttaaacaagacttccgagtgtgtgtttgtgtatttggttgagacttgaggagagacacagaggaggatgggcttttttgtttcctctctgcttagtactcatatcctatcattattattattactactacttattattgaaaccctcgcttatgtagtggttttgatttagggttaggattgcaccaaaaataagatccactttaccacttagtcttgctcataagtacgatgaagaacatttaattagcttctcttcttgtcattgtaagctacctacacatttctgatctttatcaagatactactacttttc

SEQ ID NO: 3 - DNA, 1833 bp, Arabidopsis thaliana
  CDS 113...1612
  ref <301>: AUTHORS: Tomohiro Kiyasue, Yoshu Yoshiba, Kazuko Yamaguchi- Shinozaki, Kazuo Shinozaki
  ref <302>: TITLE: Title : A nuclear gene encoding mitochondrial prolne dehydrogenase, an enzyme involved in proline metabolism, is upregulated by proline but downregulated by dehydration in Arabidopsis.
  ref <303>: JOURNAL: The Plant Cell
  ref <304>: VOLUME: 8
  ref <306>: PAGES: 1323-1335
  ref <307>: DATE: 1996-05-27
  ref <308>: DATABASE ACCESSION NUMBER: D83025
  ref <309>: DATABASE ENTRY DATE: 1995-12-25
agcgtttagaaaaaaacagcgataaaaccgaaacatcaagcaaacaaaaaaaaaagagaagagaaattatttttttttgttttcgttttcaaaaacaaaatctttgaattttatggcaacccgtcttctccgaacaaactttatccggcgatcttaccgtttacccgcttttagcccggtgggtcctcccaccgtgactgcttccaccgccgtcgtcccggagattctctcctttggacaacaagcaccggaaccacctcttcaccacccaaaacccaccgagcaatctcacgatggtctcgatctctccgatcaagcccgtcttttctcctctatcccaacctctgatctcctccgttccaccgccgtgttgcatgcggcggcgataggtcctatggtcgacctagggacgtgggtcatgagctctaaacttatggacgcttcggtgacgcgtggcatggttttagggcttgtgaaaagtacgttttatgaccatttttgcgccggtgaagatgccgacgcagccgctgagcgcgtgagaagcgtttatgaagctactggtcttaaagggatgcttgtctatggcgtcgaacacgccgatgacgctgtatcttgtgatgataacatgcaacaattcattcgaaccattgaagctgccaaatctttaccaacatctcactttagctcagtggttgtgaagataactgccatttgtccaattagtcttctgaaacgagtgagcgatctgctgcggtgggaatacaaaagtccgaacttcaaactctcatggaagctcaaatcgtttccggttttctccgaatcgagtcctctctaccacacaaactcagaaccggaaccgttaaccgcggaagaagaaagggagctcgaagcagctcatggaaggattcaagaaatctgtaggaaatgccaagagtccaatgtaccattgttgattgatgcggaagacacaatcctccaacccgcgatcgattacatggcttattcatcggcgatcatgttcaatgctgacaaagaccgaccaatcgtttacaacacgattcaggcgtacttgagagacgccggtgagagactgcatttggcagtacaaaatgctgagaaagagaatgttcctatggggttcaagttggtgagaggggcttacatgtctagcgaacgtagcttggcggattccctgggttgcaagtcgccagtccacgacacaattcaggatactcactcttgttacaatgattgtatgacattcctgatggagaaagcatcaaacggttctggtttcggtgtcgttctcgcaacacataacgctgattcggggagacttgcgtcgaggaaagcgagtgacctcgggatcgataaacagaacgggaagatagagtttgcacagctatatggtatgtcagatgcattgtccttcgggttaaagagagcagggttcaatgttagcaagtacatgccgtttggacccgtcgcaaccgctataccgtatcttctccgacgcgcttatgagaaccggggaatgatggccaccggagctcatgaccgtcaactcatgaggatggaacttaagaggagattaatcgccgggattgcgtaaagagagagtatggagccattaaatgaaattgggaaatgtagatgaataaatttcttctatgtagtttaagaaattgaaaacaaaaaattataatataagaaatggagtaggtaagaacatttcctgtggctaaatatttttcatgagggactatgtttttactatcaatatatcattcacaaatgtatattcaccttatcaataaaaatgctttttacttt